(19) United States
(12) Patent Application Publication
(10) Pub. No.: US 2011/0184249 A1
(43) Pub. Date: Jul. 28, 2011

(54) REMOTE PATIENT MONITORING SYSTEM

(76) Inventor: DANIEL C. DAVIS, JR., Honolulu, HI (US)

(21) Appl. No.: 13/008,754

(22) Filed: Jan. 18, 2011

Related U.S. Application Data
(60) Provisional application No. 61/282,345, filed on Jan. 27, 2010.

(51) Int. Cl. G06Q 50/00; A61B 5/00

(52) U.S. Cl. 600/300; 705/2

(57) ABSTRACT

The remote patient monitoring system includes a camera and radio connection device having electronic circuitry disposed in a housing. The device facilitates connection of interchangeable cameras and reconfigurable radios for remote monitoring of visually observable health indicia of a patient by a remotely located health professional. The system may also include a remote server for management of a plurality of patient sites and monitoring devices. The patient health related video images are digitally transmitted from the patient site to the remote health care provider over the Internet.

### CROSS-REFERENCE TO RELATED APPLICATION

This application claims the benefit of U.S. Provisional Patent Application Ser. No. 61/282,345, filed Jan. 27, 2010.

### BACKGROUND OF THE INVENTION

1. FIELD OF THE INVENTION

The present invention relates generally to health monitoring systems, and more particularly to a remote patient monitoring system having a device that facilitates a variety of cameras and wireless transmission means at a patient site.

2. DESCRIPTION OF THE RELATED ART

Remote patient monitoring systems allow the transmission of a patient's physiological data from their home to a health care professional at a remote location over a communications network. Videoconferencing may be used, in which the patient and health care professional are connected audio-visually over a telephone line or other suitable two-way communications channel. In this manner, teleconference can be used to check up on patient recovery progress, verify medication compliance, illustrate to a patient how to perform home care, and the like.

The problem is that these systems are often inconvenient, inefficient, or simply not able to perform patient monitoring tasks well. For example, some camera systems are inadequate because they use digital zoom, which may cause pixelation of the image when zooming. Moreover many of these camera systems only provide 2× power, which may not be adequate enough to visually resolve clinical details, such as skin lesions, neck vein distension, pupillary light reflexes, and the like.

Additionally, existing systems typically have multiple wire connections, with no integrated cable management offered. Such wired connections are easily tangled, unsightly, not user friendly, often obtrusive, and lack discretion and privacy. The existing system devices are generally fixed components, and therefore cannot be tailored to individual patients and their data communication setup, nor are these systems adaptable to changing medical conditions of the individual patients.

Another disadvantage is that existing systems may not be battery powered, thus generally requiring them to be plugged into the wall to provide a power source. This is undesirable since it limits the unit's portability and safety, possibly rendering the device in violation of IEC 60601 medical electrical equipment standards.

In addition, many existing remote patient monitoring systems require the patient or visiting nurse to have an Internet-ready computer. This places an undue burden on the patient and visiting health care professional. Other disadvantages may include requiring the camera to be hard-wired to the patient's computer. It is readily appreciated that such a configuration limits where the camera can be positioned, which may frustrate a remote operating clinician's attempt to get a closer look at that rash, and the like. In current IP-based communications systems, a patient or caregiver may have to manually log into a client's computer or gateway located at the patient's site, thus further limiting ease of use of the system.

Thus, a remote patient monitoring system solving the aforementioned problems is desired.

### SUMMARY OF THE INVENTION

The remote patient monitoring system includes a device having electronic circuitry disposed in a rectangular or other suitable housing. The device facilitates connection of interchangeable cameras and interchangeable radios for remote monitoring of visually observable health indicia of a patient by a remotely located health professional. The system may also include a remote server for management of a plurality of patient sites and monitoring devices. The patient health-related video images are digitally transmitted from the patient site to the remote health care provider over the Internet.

The system provides a simple, effective, high quality telemedicine video and audio link between a remote patient and a clinical consultant. The system is configured so that, on the patient side, all that is required is the pressing of a single on-off button to activate patient monitoring. Moreover, on the patient side, required system components are battery-operated. Content delivery is preferably wireless. The patient “Examination Camera” is preferably Internet-ready, and in combination with a wireless radio transceiver, can provide high quality real-time streaming digital video and auscultation from the remote patient to the consulting clinician.

These and other features of the present invention will become readily apparent upon further review of the following specification and drawings.

### BRIEF DESCRIPTION OF THE DRAWINGS

FIG. 1 is a front perspective view of a telemedicine adapter box used in a remote patient monitoring system according to the present invention.

FIG. 2 is a rear perspective view of the telemedicine adapter box of FIG. 1.

FIG. 3 is a screenshot of a login web page used in a remote patient monitoring system according to the present invention.

FIG. 4 is a screenshot of the available examination cameras web page in a remote patient monitoring system according to the present invention.

FIG. 5 is a screenshot of an exemplary remote camera viewing and control web page in a remote patient monitoring system according to the present invention.

FIG. 6 is a block diagram of the telemedicine adapter box used in a remote patient monitoring system according to the present invention.

FIG. 7 is a perspective view of the internal layout of a portion of a telemedicine adapter box used in a remote patient monitoring system according to the present invention.

FIG. 8A is a block diagram showing a first portion of the process flow of a remote patient monitoring system according to the present invention.

FIG. 8B is a block diagram showing a second portion of the process flow of a remote patient monitoring system according to the present invention.

FIG. 9 is a block diagram of a remote patient monitoring system according to the present invention.

Similar reference characters denote corresponding features consistently throughout the attached drawings.

### DETAILED DESCRIPTION OF THE PREFERRED EMBODIMENTS

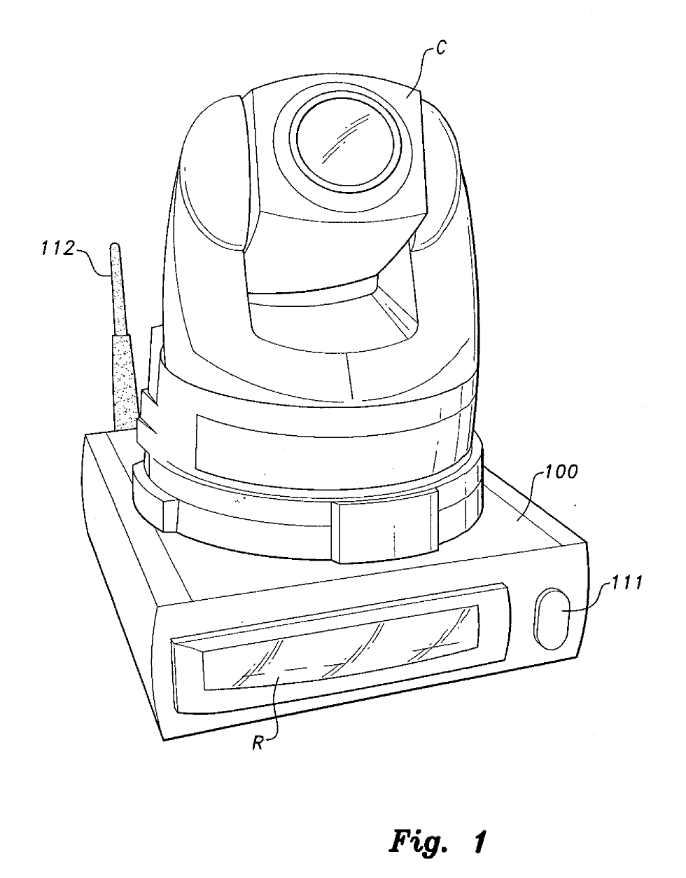

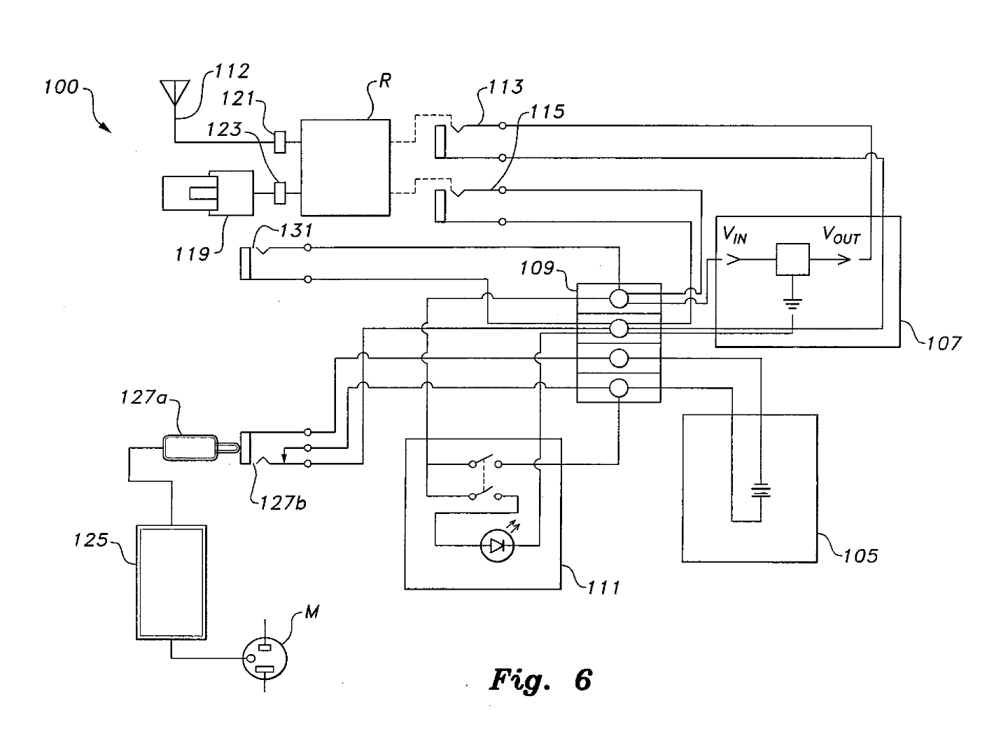

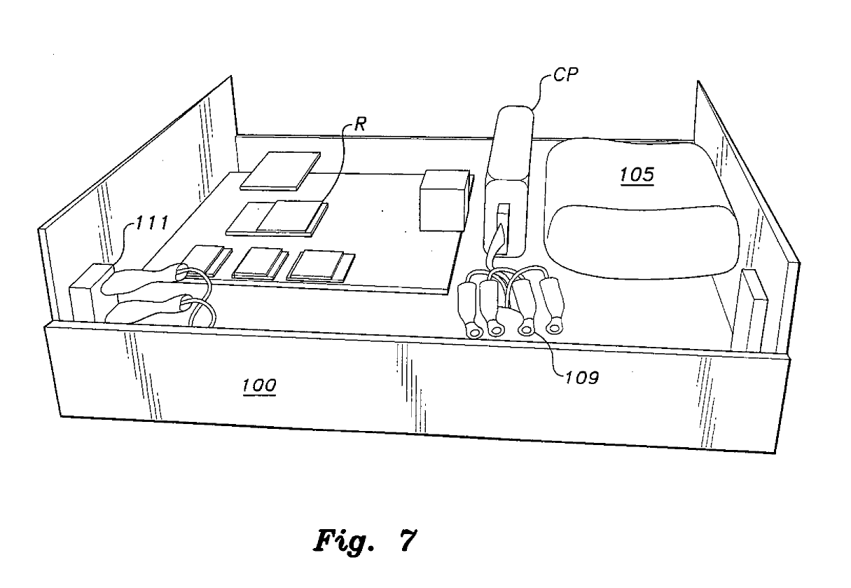

As shown in FIGS. 1 and 6, the remote patient monitoring system includes a device having electronic circuitry disposed in a compact, portable, rectangular housing 100. The portable device portion 100 of the system is located at a patient site for monitoring of the patient. The system facilitates connection of interchangeable cameras and interchangeable radios, such as camera C and radio R, for remote monitoring of visually observable health indicia of the patient by a remotely located health professional using a network connection, such as a plain old telephone system (POTS), Integrated Services Digital Network (ISDN), Data Over Cable Services Interface System (DOCSIS), the Internet, or the like. Moreover, as shown in FIG. 7, an interchangeable 3G, 4G, or next generation cellular phone CP may be connected to the system. Preferably, the cellular phone CP is an embedded, board level radio that has pluggable data and power connectors. Any radios R of the housing 100 are connected to a suitable antenna 112, which is disposed in the external rear portion of the housing 100.

Preferably, the camera C is a pan, tilt, zoom (PTZ) camera in which the clinician remotely controls the camera's video (pan, tilt, zoom, lighting, snapshots, etc) through a simple, intuitive browser interface. The camera C preferably is addressable using Internet Protocol, and also features 2-way audio, the streaming video feed and audio feed being accessible by a web browser.

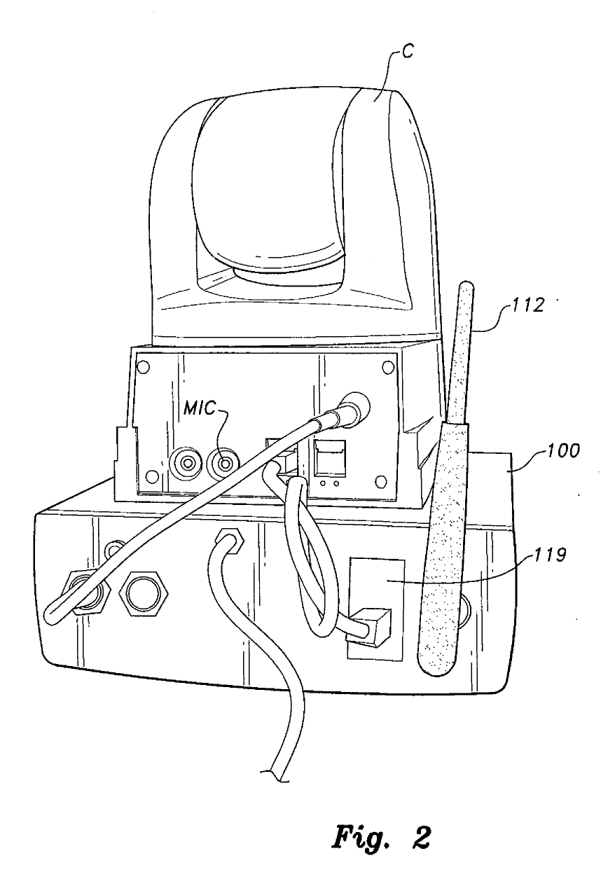

While the camera C is illustrated as being positioned on top of a telemedicine adapter housing 100, it is easily removable from the housing 100 and may be positioned near the patient or held by the patient for a close-up view of his/her observable health indicia. The camera C may include a high-quality electronic stethoscope that can be connected to the MIC receptacle (refer to FIG. 2) in the back of the camera unit C, through which the clinician can remotely listen to the heart, lungs, and other body sounds contemporaneously with the video feed of the patient. The patient and remote site caregivers turn the camera C on and off with a single button-activated switch 111. The clinician controls the video camera C and auscultation through a standard web browser. Depending on the patient's needs, the patient may have one or more devices that can be interfaced with the system for transmission of data to the remote clinician, including a digital blood pressure cuff, a digital scale, a glucose meter, a pulse oximeter, other physiologic data capture devices, a personal emergency response button, or a medication dispenser.

The wireless portion of the system may comprise a wireless card R (e.g., broadband 802.16, or 802.11/a/b/g/n) for high data throughput. To avoid having to physically swap radios, the wireless card R is preferably a software programmable (or configurable) radio that can be programmed and/or configured by software to function as 802.11 /a or /b or /g or /n, or 802.16. Moreover, wireless communication in the system may be facilitated by a cellular telephone CP. Either the wireless device R or the cellular telephone CP may singly, or in combination, provide wireless communication that supports electronic messaging, multimodal SMS, mobile phone, page, e-mail, e-fax, and transmission of HIPAA compliant digital photos (wounds, dermatology), video clips, customizable forms, best practice care management plans, best practice templates, remote vital sign collection, management, distribution, and alerting, including electronic alerting of the care team for out-of-range and missing data.

The small, electronic housing 100 includes a single large off-on button 111 as its only control. As shown in. FIGS. 6-7, a single lithium ion or other suitable rechargeable battery 105 is disposed in the housing 100 and provides power to the radio R (also disposed in the housing 100) by which the device 100 connects to the Internet.

Component docking ports are integrated into an internal printed circuit board. The IP addressable digital video camera C contains a web server and software, which delivers a specially configured medical use web page to a requesting web browser. Different IP addressable PTZ video cameras can be substituted to meet the requirements of the clinical situation. A CAT 5, RJ-45, or other suitable connector 119 disposed in the rear portion of the housing 100 provides a data port, which allows the PTZ video camera C to connect to a wireless bridge/router, e.g., data radio R, via a short network patch cable. The camera C is powered by the lithium ion battery 105 via a voltage regulating/reducing module 107 when the switch 111 is turned on.

The radio R, being enclosed within the electronics housing 100, is modular so that the enclosed radio R can be quickly changed or reprogrammed to meet local communication requirements, such as 802.11 a/b/g/n or broadband cellular, such as 3G or 4G cellular broadband. The radio R is powered by the lithium ion battery 105 via the voltage regulating module 107, which is designed to be compatible with the specific radio R being used. The power connection of battery 105 to system components is facilitated through a power buss 109, which distributes power to a camera power connector 131, a first radio power connector 113, and a second radio power connector 115. The first radio power connector 113 receives reduced voltage power from the output of voltage regulator/reducer 107 and is used when the radio R requires only 5 volts to operate. The second radio power connector 115 receives full battery voltage directly from the battery 105, and is generally used when the radio R requires 12 volts to operate. Preferably the connectors 113, 131, and 115 are of the quick connect variety.

An antenna connector 121 and a data connector 123 are disposed in the housing 100. The antenna connector 121 removably connects the antenna 112 to the radio R. The data connector 123 removably connects the RJ-45 data connector to the radio R. Both are quick connect type connectors to facilitate easy connection and removal of the radio R. A camera power connector 131 is connected to the voltage regulator/reducer 107 via the power buss 109 so that the voltage delivered to the camera C is selectable to either 5 volts or 12 volts, depending on the type of camera being connected to the camera power connector 131.

To keep the battery 105 at optimum charge, a battery charger 125 connectable to the power mains M is provided. The output of the battery charger 125 is connected to a connector plug 127a, which can be received by a charging receptacle 127b disposed in the housing 100. The charging receptacle 127b is connected through the buss 109 to the battery 105 so that when the plug 127a engages the receptacle 127b, power is removed from components of the system, except for charge current being delivered to the battery 105 via the charger 125. This feature ensures that the system device 100 is in full compliance with International Electrotechnical Commission (IEC) 60601 medical electrical equipment standards.

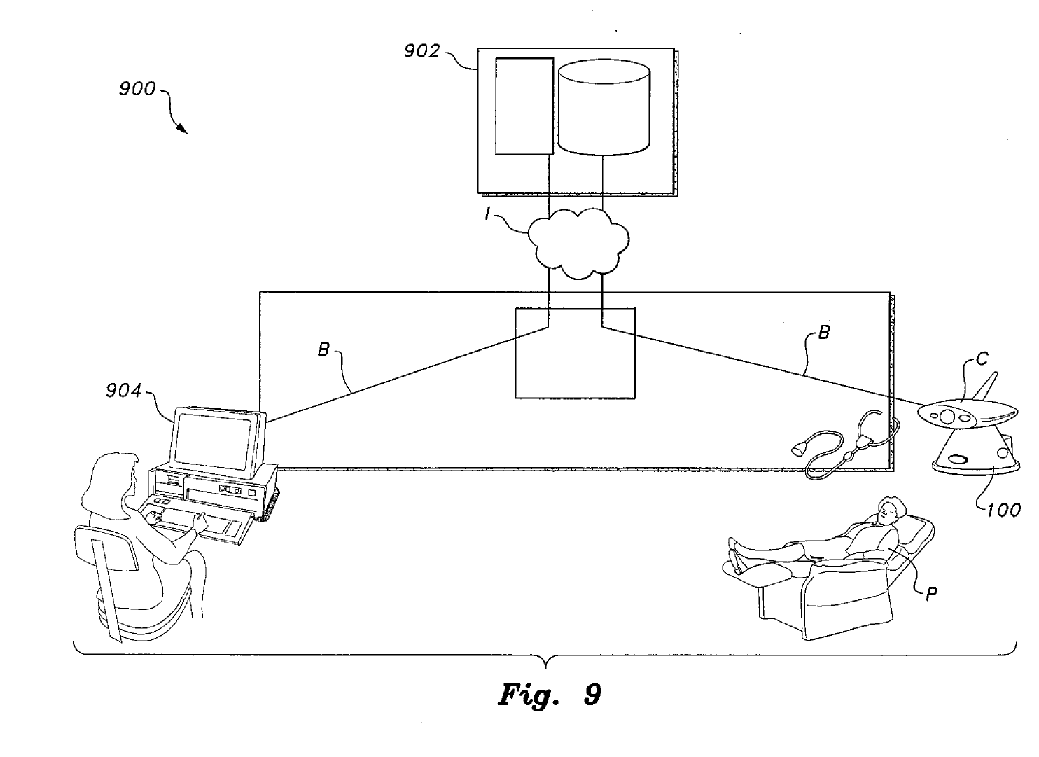

As shown in FIG. 9, the system may also have a delivery system 900 that includes a broadband connection B via the Internet I to a remote server-mass storage unit 902 for management of a plurality of patient sites and monitoring devices C connected to the housing 100. Clinician workstations 904 are also connected to the system via a broadband connection B. The health-related video images of the patient P are digitally transmitted from the patient site to the remote health care provider over the Internet, the process being mediated by at least one server-mass storage unit 902. Preferably, the server 902 comprises an application service provider (ASP) delivered software system that manages communication privacy and security. The consulting clinicians connect to the camera C (“Examination Camera”) using a PC-based web browser and an encrypted Internet connection through a web software application delivered from an ASP (application service provider) server that manages permissions, security, and HIPAA compliance.

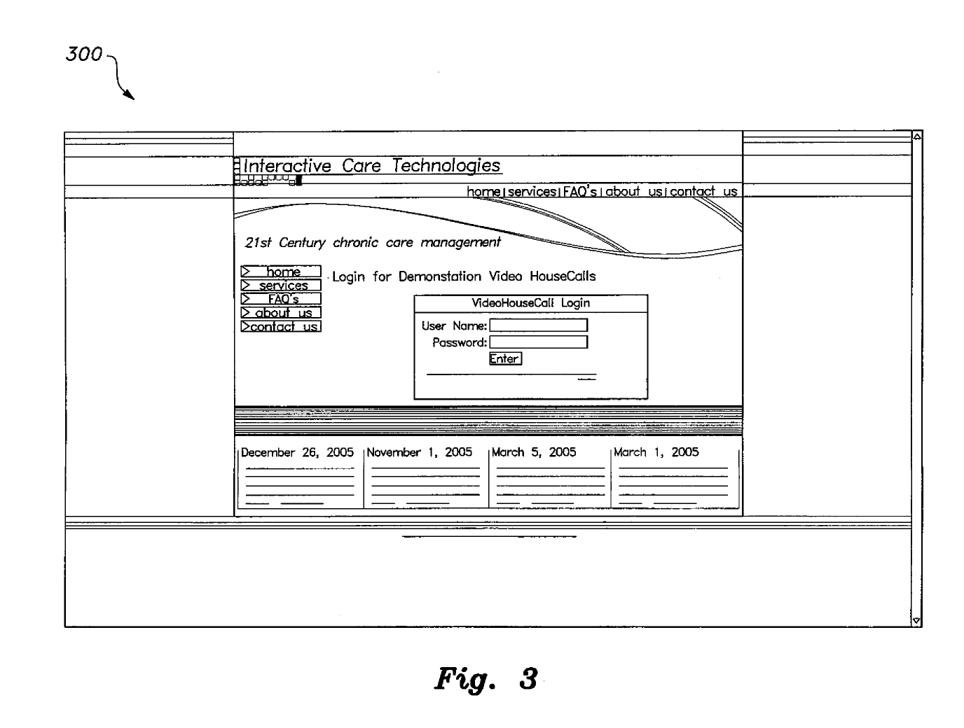

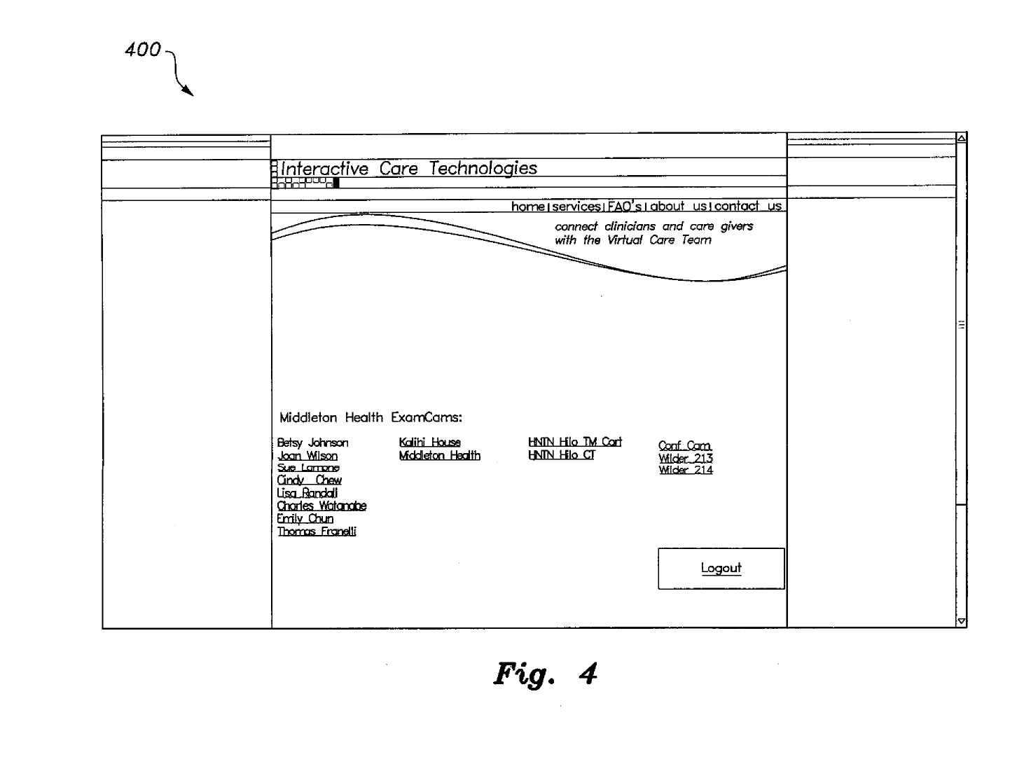

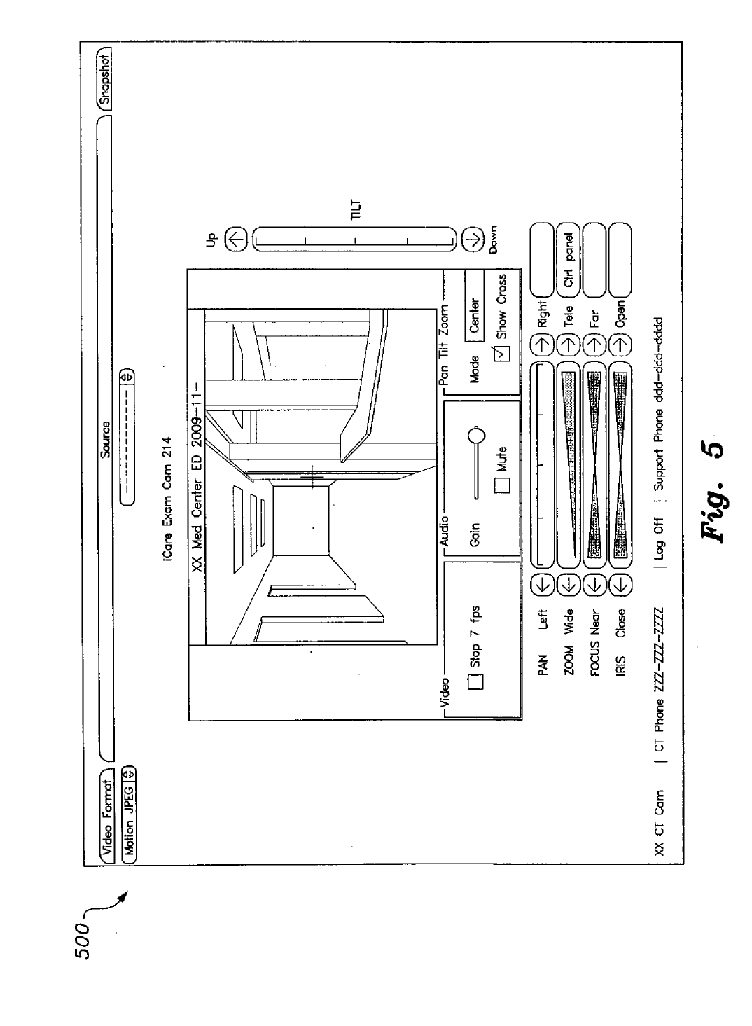

As shown in FIGS. 3, 4, and 5, the web pages provided by the ASP server include a login page 300, a remote camera list page 400, and a remote camera viewing and control page 500. The list page 400 allows the clinician to click on a patient name to activate that patient's remote camera. If the server 902 determines that the camera is unavailable for remote transmission, the patient's name may be grayed out. The remote camera viewing and control page 500 allows the clinician to do camera stills, as well as pan, tilt, zoom and focus the remote camera C. A patient ID or other identifier is displayed in the Source field at the top central portion of the web page 500.

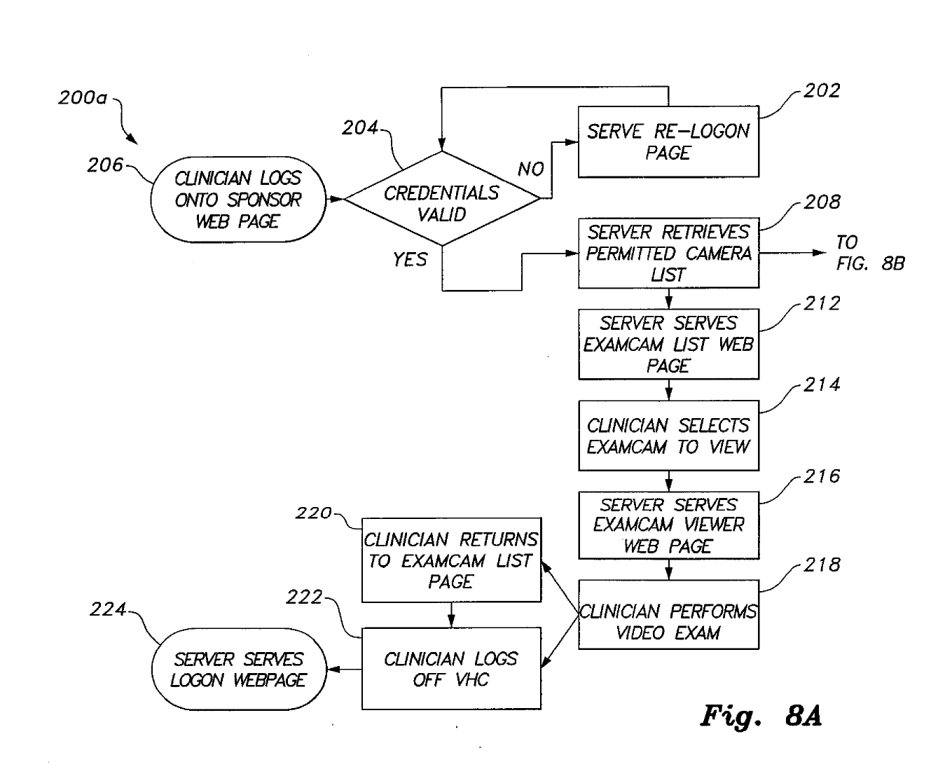

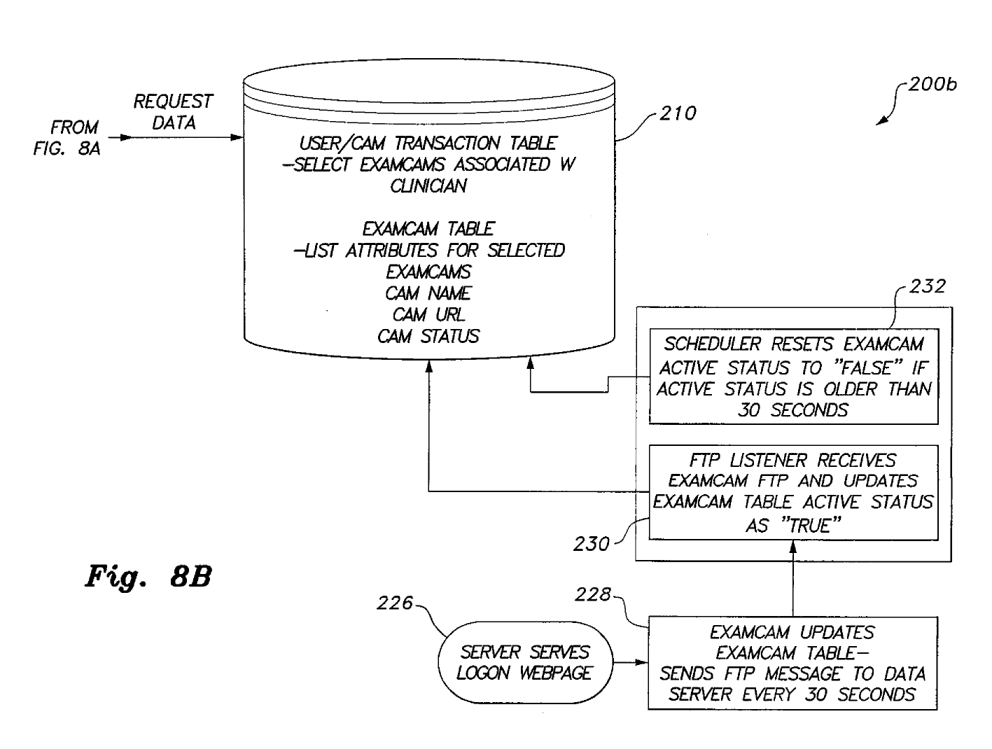

As shown in FIGS. 8A-8B, workflow on the side of the server 902 provides a secure clinician login via steps 206, 204 and 202. Credentials must be successfully validated before the server 902 retrieves any of the confidential, Health Insurance Portability and Accountability Act (HIPAA) related patient data. The server then retrieves a permitted camera list at step 208 by requesting camera data from a user/camera transaction table, a clinician camera association table, and an Examination Camera attributes table from camera data store 210. Camera attributes include camera ID's, names associated with the camera ID's, URL's associated with the cameras, and statuses of the cameras being retrieved by the clinician.

Once the camera data has been retrieved, the Server populates the list page 400 and serves the list page 400 to the clinician's web-enabled browser. At step 214, the clinician clicks on a particular patient name to select that patient's Examination Camera for viewing. At step 216, the server 902 responsively makes the Examination Camera viewing and control page 500 available. Utilizing the controls available on the camera viewing and control page 500, the clinician performs the video examination of the patient P at step 218, after which the clinician has the choice of either selecting (at step 220) another patient on the list page 400, or merely logging off the system from any page on the system. At step 224, the server 902 then makes available the log on page 300 for later clinician entry into the system.

Features of the system in combination with delivery system 900 include scalable home physiologic monitoring through the telephone; automated home medication administration and compliance monitoring; flexible personal emergency response system; 24-by-7 triage call centers; an anywhere, anytime web-based clinician interface; and intelligent e-messenger with cascading alerts, low-cost entry, and ASP web architecture. File Transfer Protocol or other suitable message communication to patient cameras C in the system may be periodically initiated by the server 900 to determine the status and availability of the cameras C in order to update the camera list web page 400.

It is to be understood that the present invention is not limited to the embodiment described above, but encompasses any and all embodiments within the scope of the following claims.

I claim:

1. A remote patient monitoring system, comprising:
an interchangeable electronic camera;
a reconfigurable data radio;
a compact, portable enclosure;
an electronic circuit disposed in the compact portable enclosure, the electronic circuit having a rechargeable battery, the interchangeable electronic camera and the reconfigurable data radio being removably attachable to the compact portable enclosure;
an illuminated on-off switch;
a power buss connected to the rechargeable battery, the power buss selectively distributing battery voltage to the electronic circuit, the reconfigurable data radio, and the interchangeable camera through the illuminated on-off switch;
a battery charging receptacle connected to the power buss, the battery charging receptacle providing charging current to the battery while disconnecting the battery from the electronic circuit when a battery charger is connected to the battery charging receptacle;
a camera power connector;
first and second radio power connectors, the second radio power connector receiving full battery voltage from the rechargeable battery and applying full battery voltage to the data radio when the data radio is connected to the second radio power connector;
a radio data connector removably connecting the electronic camera to the data radio;
a radio antenna connector for removably connecting an antenna to the data radio; and
a voltage-reducing circuit for reducing voltage supplied by the battery, the voltage-reducing circuit selectively applying a reduced voltage to the camera through the camera power connector and applying the reduced voltage to the data radio through the first radio power connector;
wherein, when the camera is trained on a patient, the radio securely sends video information about visually observable indicia of the patient's health to a remotely located health professional.

2. The remote patient monitoring system according to claim 1, further comprising means for high speed distribution of patient health data to the remotely located health professional.

3. The remote patient monitoring system according to claim 1, further comprising means for storing management and availability information regarding a plurality of said cameras located in a corresponding plurality of patient sites, the availability information including camera ID's names associated with the camera ID's, URL's associated with the cameras, and statuses of the cameras being accessed by the remotely located health professional.

4. The remote patient monitoring system according to claim 1, further comprising means for managing permissions, security, and HIPAA compliance associated with the patient health data.

5. The remote patient monitoring system according to claim 1, wherein said voltage reducer is a voltage regulator circuit.

6. The remote patient monitoring system according to claim 1, wherein said first and second radio power connectors are quick-connect connectors.

7. The remote patient monitoring system according to claim 1, wherein said radio data connector and said radio antenna connector are quick-connect connectors.

8. The remote patient monitoring system according to claim 1, further comprising means for performing still photo, pan, tilt, zoom and focus operations in the camera responsive to commands issued by the remotely located health professional.

9. The remote patient monitoring system according to claim 1, further comprising means for electronic message delivery of said patient health data to remote users, said message delivery including cascading alerts.

10. The remote patient monitoring system according to claim 9, wherein said means for electronic message delivery further comprises means for multimodal SMS, mobile phone, page, e-mail, e-fax electronic messaging of HIPAA compliant digital photos, video clips, customizable forms, best practice care management plans, best practice templates, remote vital sign data, and out-of-range and missing data electronic alerts to a care team of users.

11. The remote patient monitoring system according to claim 9, wherein said camera includes a high-quality electronic stethoscope transmitting an auscultation patient health data.

12. A remote patient monitoring system, comprising:
means for retrieving patient data transmitted from a remote source, the patient data including patient video and patient health data;
means for credential check and logon of a health professional user to a secure web page associated with the remote patient monitoring system;
means for periodically determining status and availability of the patient video and health data remote source; and
means for displaying the patient video and health data on the web page;
wherein the health professional user can perform a remote video examination of a patient associated with the patient data.

13. The remote patient monitoring system according to claim 12, further comprising means for high speed transmission of tilt, pan, zoom, focus, and still video control data to the remote source responsive to commands issued by the health professional user.

14. The remote patient monitoring system according to claim 12, further comprising means for displaying management and availability information regarding a plurality of said remote data sources located in a corresponding plurality of patient sites, the availability information including video source ID's, names associated with the video source ID's, URL's associated with the video sources, and statuses of the video sources being accessed by the health professional user.

15. The remote patient monitoring system according to claim 12, further comprising means for displaying electronic messages associated with the patient health data to the health professional user, means for displaying electronic messages including means for displaying cascading alerts.

16. The remote patient monitoring system according to claim 15, wherein said means for displaying electronic messages further comprises means for displaying multimodal SMS, mobile phone, page, e-mail, e-fax electronic messaging of HIPAA compliant digital photos, video clips, customizable forms, best practice care management plans, best practice templates, remote vital sign data, and out-of-range and missing data electronic alerts to a care team of health professional users.

17. The remote patient monitoring system according to claim 15, further comprising means for presenting high-quality electronic stethoscopic auscultation patient health data transmitted by said remote data source to the health professional user.
